(19) United States
(12) Patent Application Publication
(10) Pub. No.: US 2019/0021959 A1
(43) Pub. Date: Jan. 24, 2019

(54) AQUEOUS DISPERSED COLOR COMPOSITION WITH HIGH PIGMENT CONTENT AND AQUEOUS NAIL POLISH COMPOSITION USING THE SAME

(71) Applicant: Chemipams.Co.Ltd, Incheon (KR)

(72) Inventor: Hyoung jin Min, Incheon (KR)

(21) Appl. No.: 16/040,420

(22) Filed: Jul. 19, 2018

(30) Foreign Application Priority Data
Jul. 20, 2017 (KR) 10-2017-0091931

(51) Int. Cl. A61K 8/04; A61K 8/81; A61K 8/87; A61Q 3/02

(52) U.S. Cl. CPC A61K 8/04; A61K 8/8152; A61K 8/8117; A61K 8/87; A61K 2800/805; A61K 2800/43; A61K 2800/412; A61K 2800/524; A61Q 3/02

(57) ABSTRACT

The present invention relates to an aqueous dispersed color composition with a high pigment content and an aqueous nail polish composition using the same, and more particularly, to an aqueous dispersed color composition containing an alkaline resin aqueous solution including an alkali-soluble resin having a weight-average molecular weight of 3,000 to 20,000 and an aqueous dispersed emulsion resin having a weight-average molecular weight of 100,000 to 300,000, so that the aqueous dispersed color composition stably contains a high pigment content in a dispersed state, no color deformation, occur in a pigment, and dispersion stability and color expression of the pigment are excellent. In addition, an aqueous nail polish composition using the aqueous dispersed color composition is easily mixed with pigments, and the aqueous nail polish composition has excellent gloss property, color expression, adhesion property, and storage stability even when stored for a long period of time.

### CROSS-REFERENCE TO RELATED APPLICATION

This application claims the benefit of Korean application No. 10-2017-0091931, filed on Jul. 20, 2017, the disclosure of which is incorporated herein by reference.

### BACKGROUND OF THE INVENTION

### 1. Field of the Invention

The present invention relates to an aqueous dispersed color composition with a high pigment content and an aqueous nail polish composition using the same.

### 2. Description of the Related Art

Recently, the nail field has been remarkably developing in beauty industries, so the distribution and interest for nail-related products have been accelerated in the public. Conventionally, the nail-related products have been developed in consideration of beauty other than health, and oil nail polish is the most generally used product among the nail-related products.

The oil nail polish contains various components, in which main components are organic solvents, such as toluene, n-butylacetate, ethylacetate, and methylethylketone, and endocrine disrupters such as dibutylphthalate (DBP) and dioctylphthalate (DOP). The components of the oil nail polish are known to provide stimulation to skin, eyes, nose, and neck, and affect a central nervous system.

In particular, the organic solvents may be absorbed through the skin in the form of liquid or gas, and mainly diffused in the air in the form of gas and inhaled through respiratory organs to primarily stimulate mucous membranes of the eyes, skin, and respiratory organs. In addition, the organic solvents affect functions of the central nervous system, causing central nervous system depression symptoms such as headache, dizziness, nausea, and euphoria. Further, it has been reported that long-term exposure of the organic solvents causes chronic disorders in the functions of the central nervous system even at low concentrations, resulting in cognitive and emotional disorders such as fatigue, paresthesia, hypomnesis, and confusion (Douglas et al., 1986; Gyu-sang Jo, 1991).

In order to improve skin side effects of the conventional oil nail polish, an aqueous nail polish product using water as a medium has been released. The conventional aqueous nail polish has been using pigments that have water-soluble groups (carboxyl group, sulfonic acid group, hydroxyl group, etc.) so as to be easy to dissolve in the water or using surfactants having a highly hydrophilic property, that is, a hydrophilic-lipophilic balance (HLB) of 10 to 20 to disperse the pigments.

The pigments used in the conventional aqueous nail polish have hydrophilicity on surface of particles so as to be easily dispersed in a water phase, while the pigments are colored on fingernails (nail plates) when the nail polish is used. In addition, when the pigments are dispersed using the surfactant having a high HLB, a large amount of foam is generated during preparation of a product, and affinity of the surfactant with respect to water and oil varies depending on temperature, thereby degrading stability of the dispersed pigments.

The aqueous nail polish has been developed focusing on improving completeness of a base composition (that is, a composition before the pigments are included) up to now. When the pigments are mixed with the base composition to prepare the aqueous nail polish composition, color separation and layer division may occur in the composition, or the gloss property and color expression may be degraded. In addition, pigment particles are re-agglomerated as the time elapses, and fingernail adhesion property and storage stability of the aqueous nail polish may be degraded.

Generally, the pigments used in the nail polish have different oil absorption values, so that, in order to overcome oil absorption deviation between the pigments contained in the nail polish, an excessive amount of surfactants or dispersants is used;, or a small amount of pigment is used. The nail polish in which an excessive amount of surfactants or dispersants is used may cause degradation in water resistance and adhesion property of the nail polish due to the excessive amount of surfactants or dispersants, or may stimulate the skin and fingernails. In addition, when a small amount of pigments is contained in the nail polish, the color expression and coloring strength of the nail polish may be degraded.

When a particle size of the nail polish pigment becomes smaller, the color expression and coloring strength become more excellent, whereas a surface area and surface energy of the pigment are increased so that the pigment particles become unstable and form a secondary particle by coagulation and condensation. In addition, dispersibility of the pigments is degraded due to the coagulation and condensation of the pigment particles, and finally, color separation or layer division may occur in the composition containing the pigments, thereby degrading the storage stability of the composition.

Korean Patent Application Publication No. 10-2011-0062438discloses a related art on an aqueous nail polish, which relates to a coating film-separable aqueous nail polish composition. The related art does not use the plasticizers and organic solvents, which are components of the conventional oil nail polish, to improve the stimulation by organic solvents and plasticizers. However, the pigments contained in the nail polish composition are separated as the time elapses.

Korean Patent Application Publication No, 10-2012-0039080 discloses another related art on the aqueous nail polish, which relates to an aqueous nail polish composition using an aqueous resin solution having a glass transition temperature of −5° C. to 30° C. In the related art, the glass transition temperature of the aqueous resin solution is low, so that it is easy to form a film on the fingernails by applying the nail polish composition to the nails, whereas a dried film is insufficient in the water resistance and heat resistance, causing a whitening phenomenon when making contact with the water or causing the film to be easily separated off.

Recently, as domestic consumption of the nail polish has increased due to the boom-up of nail art in Korea, demands of consumers for a human body-friendly nail polish have increased, so that the aqueous nail polish using the water as a medium has been developed. However, research and product development for the aqueous nail polish have not been actively performed.

### SUMMARY OF THE INVENTION

Accordingly, to solve the problems of the oil and aqueous nail polishes, an object of the present, invention is to provide a method of preparing an aqueous dispersed color composition with a high pigment content.

In addition, an object of the present invention is to provide an aqueous dispersed color composition with a high pigment content and an aqueous nail polish composition using the same.

To achieve the objects described above, according to the present invention, there is provided a method of preparing an aqueous dispersed color composition with a high pigment content, the method including: a step (S10) of preparing an alkaline resin mixture by mixing water with an alkali-soluble resin; a step (S20) of preparing an alkaline resin aqueous solution by aging the alkaline resin mixture in the step (S10); a step (S30) of preparing a first pigment mixture by dispersing and emulsifying a pigment in the alkaline resin aqueous solution in the step (S20); a step (S40) of preparing a second pigment mixture by mixing an aqueous dispersed emulsion resin with the first pigment mixture in the step (S30); a step (S50) of preparing a particulate dispersion by particulating and dispersing the second pigment mixture in the step (S40) with a milling machine; and a step (S60) of filtering the particulate dispersion in the step (S50) with a housing filter.

In addition, according to the present invention, there is provided an aqueous dispersed color composition and an aqueous nail polish composition using the same.

The aqueous dispersed color composition with the high pigment content and the aqueous nail polish composition prepared according to the present invention do not cause skin irritation and has excellent storage stability, excellent expression and gloss property, no color deformation, and excellent adhesion property.

In addition, the aqueous dispersed color composition with the high pigment content and the preparation method thereof according to the present invention allows pigments to be easily mixed into the aqueous nail polish composition, so that various aqueous nail compositions can be easily prepared, thereby improving productivity.

### BRIEF DESCRIPTION OF THE DRAWINGS

FIG. 1 is a flowchart for explaining a method of preparing an aqueous dispersed color composition with a high pigment content according to the present invention.

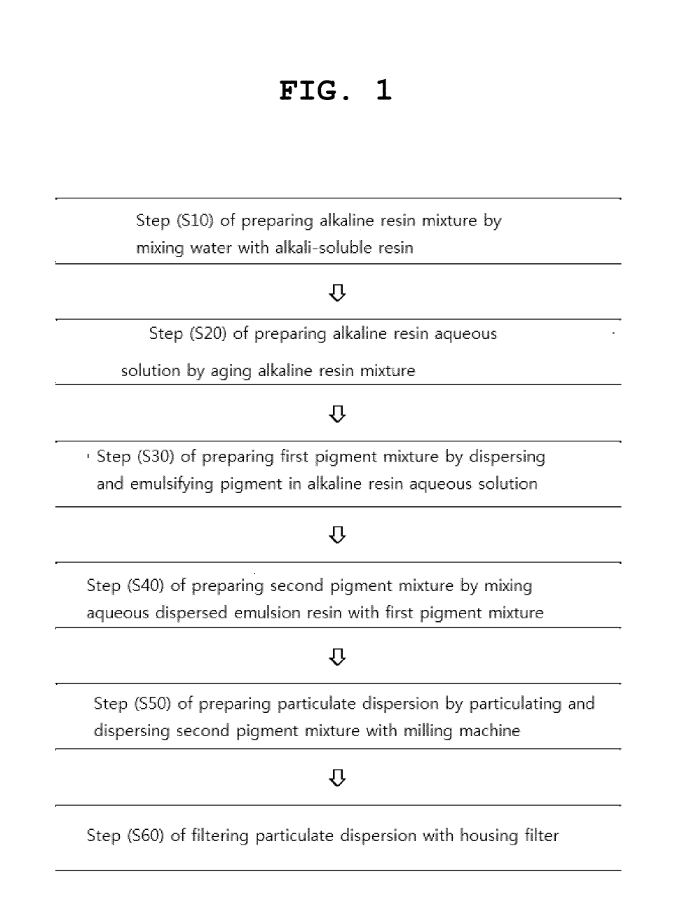

FIG. 2 is a view for explaining the adhesion property of an aqueous nail polish composition according to one embodiment of the present invention.

FIG. 3 is a view for explaining the storage stability of the aqueous nail polish composition according to one embodiment of the present invention.

### DETAILED DESCRIPTION OF THE INVENTION

Hereinafter, the present invention will be described in detail.

In the present invention, water may be selected from purified water, distilled water, and ion-exchange water.

According to the present invention, a method of preparing an aqueous dispersed color composition with a high pigment content includes: a step: S10 of preparing an alkaline resin mixture by mixing water with an alkali-soluble resin; a step S20 of preparing an alkaline resin aqueous solution by aging the alkaline resin mixture in the step S10; a step S30 of preparing a first pigment mixture by dispersing and emulsifying a pigment in the alkaline resin aqueous solution in the step S20; a step S40 of preparing a second pigment mixture by mixing an aqueous dispersed emulsion resin with the first pigment mixture in the step S30; a step S50 of preparing a particulate dispersion by particulating and dispersing the second pigment mixture in the step S40 with a milling machine; and a step S60 of filtering the particulate dispersion in the step S50 with a housing filter.

Hereinafter, the method of preparing the aqueous dispersed color composition with the high pigment content according to the present invention will be described in detail.

First, the step S10 of preparing the alkaline resin mixture by mixing the water with the alkali-soluble resin is performed. The step S10 is a step of dispersing the alkali-soluble resin in the water, in which the alkali-soluble resin is mixed and dispersed while maintaining a temperature of a double jacket, reactor containing the water, glycols, and a pH regulator at 30° C. to 40° C. At this time, it is preferable that the alkali-soluble resin is introduced and dispersed into the water in a dropping scheme, and a dropping time is preferably within 2 hours,

In the step S10, the alkaline resin mixture may be prepared by selecting one or more alkali-soluble resins. Preferably, an alkaline resin mixture having a different glass transition temperature (Tg) value can be prepared by using two kinds of alkali-soluble resins having different Tg values.

The alkaline resin mixture in the step S10 includes 20 wt % to 55 wt % of the alkali-soluble resin, 40 wt % to 75 wt % of the water, 0.1 wt % to 5.0 wt % of the pH regulator, and 1 wt % to 20wt % of the glycols based on a total weight of the alkaline resin mixture.

The alkali-soluble resin in the step S10 refers to a resin soluble in alkali, and it is preferable to use an alkali-soluble resin containing an acid group of a carboxyl group, a sulfonic acid group, or a hydroxyl group, having a glass transition temperature (Tg) of 80° C. to 170° C., and having a weight-average molecular weight (Mw) of 3,000 to 20,000. More preferably, the alkali-soluble resin may be selected from the group consisting of an acrylate copolymer resin, a styrene/maleic anhydride copolymer resin, a styrene/acrylate copolymer resin, an ammonium styrene/acrylate copolymer resin, a methylvinylether maleic anhydride resin, and an ethylene acrylic acid copolymer resin, which contain an acidic group of a sulfonic acid group or a hydroxy group, have a glass transition temperature (Tg) of 80° C. to 170° C., and have a weight-average molecular weight (Mw) of 3,000 to 20,000. When the Tg of the alkali-soluble resin is less than 80° C., the dispersion stability of the pigment is degraded, so that storage stability of the aqueous dispersed color composition and the aqueous nail polish composition using the aqueous dispersed color composition is degraded. When the Tg of the alkali-soluble resin is greater than 170° C., the aqueous nail polish composition using the aqueous dispersed color composition may have a crack after the aqueous nail polish composition is applied onto the fingernails, and the leveling of the coating film may be degraded.

Next, the step S20 of preparing the: alkaline resin aqueous solution by aging the alkaline resin mixture in the step S10 is performed. The alkali-soluble resin is completely dissolved by heating the alkaline resin mixture to 60° C. to 80° C. and maintaining the temperature for 2 hours to 5 hours, heating the alkaline resin mixture to 85° C. to 90° C., adding the pH regulator, and maintaining the temperature for 1 hour to 3 hours, and heating the alkaline resin mixture to 90° C. to 95° C. and maintaining the temperature for 1 hour to 3 hours. Thereafter, the completely dissolved resin mixture is cooled to 40° C. or lower, the pH regulator is added to adjust the pH of the contents to 8.5 to 9.0, and aging is performed at a room temperature for 1 day to 3 days to prepare the alkaline resin aqueous solution.

Next, the step S30 of preparing the first pigment mixture by dispersing and emulsifying the pigment in the alkaline resin aqueous solution in the step S20 is performed. The step S30 includes adding and dispersing the pH regulator, the water, and an antifoaming agent to the alkaline resin aqueous solution in the step S20, adding and mixing 40 wt % to 75 wt % of the pigment contained in the first, pigment mixture based on a total weight of the pigment while maintaining the temperature of the double jacket reactor at 30° C. to 40° C., adding a remaining amount of the pigment two times to four times for 1 hour in a dropping scheme, adjusting a pH to 7.0 to 8.0 by adding the pH regulator. Thereafter, the step S30 includes stirring for 3 hours while maintaining the temperature at 75° C. to 85° C. by heating an inside of the double jacket reactor, adding a preservative after cooling to 45° C. or lower, and stirring for 3 minutes to 10 minutes to prepare the first pigment mixture.

The step S30 is a step of moistening and dispersing the pigment in the alkaline resin aqueous solution, in which air and moisture present on a surface of the pigment are replacing with the alkaline resin aqueous solution to prevent the pigment particles from re-agglomerating, and to form a pigment agglomerate having an appropriate size.

The first pigment mixture in the step S30 includes 20 wt % to 40 wt % of an alkaline resin aqueous solution, 40 wt % to 75 wt % of a pigment, 0.1 wt % to 1.5 wt % of a pH regulator, 0.5 wt % to 2.5 wt % of a preservative, 0.2 wt % to 1.0 wt % of an antifoaming agent, and water as a remainder based on a total weight of the first pigment mixture.

In the step S30, the alkaline resin aqueous solution exhibits physical properties similar to physical properties of a surfactant, and serves to stabilize the moistening (wetting) of the pigment and improve the dispersion stability of the aqueous dispersed color composition. When the content of the alkaline resin aqueous solution is less than 20 wt % based on the total weight of the first pigment mixture, the content of the alkaline resin aqueous solution is insufficient, so that the pigment is not sufficiently moistened, emulsion dispersion strength and dispersion stability of the pigment are degraded, and the storage stability of the aqueous dispersed color composition is degraded. When the content of the alkaline resin aqueous solution is greater than 40 wt %, acidity of the alkaline resin aqueous solution becomes high, and the hydrophilic property becomes strong, so that it becomes difficult to perform the particulating and dispersing with a milling machine in the following step S50, and the water resistance of the aqueous dispersed color composition is degraded.

The pigment in the step S30 serves to impart color to the aqueous dispersed color composition. The pigment is preferably contained by less than 75 wt % based on the total weight of the first pigment mixture, and more preferably, 40 wt % to 75 wt %. When the content of the pigment exceeds 75 wt %, an excessive amount of the pigment is not sufficiently moistened in the alkaline resin aqueous solution, so that the alkaline resin aqueous solution hardly exhibits physical properties similar to physical properties of the surfactant with respect to the pigment, and dispersibility of the pigment is degraded. The pigment in the step S30 may be any pigment that is not harmful to a human body and an environment. Preferably, the pigment may be at least one selected from an inorganic pigment, an organic pigment, and a pearl pigment. More preferably, the pigment may be at least one selected from inorganic pigments such as mica iron oxide, white lead, red lead, iron oxide, chrome yellow, silver vermilion, ultramarine blue, Prussian blue, cobalt oxide, titanium dioxide, titanium dioxide coated, mica, titanium yellow, titanium black, iron black, molybdenum red, lithopone, emerald green, guinea green, cadmium yellow, cadmium red, and cobalt blue, organic pigments such as Red No. 104, Red No. 106, Red No. 201, Red. No. 202, Red No. 204, Red No. 220, Red No. 226, Red No. 228, Orange No. 203, Orange No. 204, Yellow No. 205, Yellow No. 401, Blue No. 1, and Blue No. 404, and pearl pigments such as mica titanium, iron oxide coated mica titanium, Prussian blue coated mica titanium, Prussian blue iron oxide coated mica titanium/chromium oxide coated mica titanium, carmine coated mica titanium, organic pigment coated mica titanium, titanium oxide synthetic mica, titanium oxide coated glass powder, iron oxide coated glass powder, and titanium oxide coated aluminum powder.

The antifoaming agent in the step S30 serves to remove air bubbles in the first pigment mixture, and may be at least one of a polysiloxane emulsion and a mineral oil emulsion.

The double jacket reactor in the step S30 may be one selected from: a double jacket reactor in which a high speed mixer is provided at left and right sides and a center of an inner upper end of the reactor, and a dissolver (Agi-mixer) is provided at an opposite end of the reactor; a double jacket reactor in which an inline mixer is provided at left and right sides of an inner lower end of the double jacket reactor, and a dissolver (Agi-mixer) is provided or fixed to left and right sides and a center of an upper end of the double jacket reactor; and a double jacket reactor in which an inline mixer is provided at left and right sides of an inner lower end of the double jacket reactor, and a dissolver coupled with a homogenizer is provided.

Next, the step S40 of preparing the second pigment mixture by mixing the aqueous dispersed emulsion resin with the first pigment mixture in the step S30 is performed. In the step S40, the aqueous dispersed emulsion resin is mixed with the first pigment mixture in which the pigment is coarsely dispersed in the step S30 and let down to prepare the second pigment mixture.

The second pigment mixture in the step S40 includes 60 wt % to 70 wt % of the first pigment mixture and 30 wt % to 40 wt % of the aqueous dispersed emulsion resin based on a total weight of the second pigment mixture.

The aqueous dispersed emulsion resin in the step S40 improves the water resistance of the aqueous dispersed color composition, and stably particulates and disperses the pigment. When the content of the aqueous dispersed emulsion resin is less than 30 wt % based on the total weight of the second pigment mixture, the water resistance and adhesion property of the aqueous nail polish composition may be degraded when; the aqueous dispersed color composition is applied to the aqueous nail polish composition. When the content of the aqueous dispersed emulsion resin is greater than 40 wt %, an excessive amount of the aqueous dispersed emulsion resin prevents the pigment from particulating and dispersing so as to cause layer division or color separation in the aqueous dispersed color composition, and a degree of gloss of the aqueous nail polish composition may be degraded.

The aqueous dispersed emulsion resin in the step S40 is preferably a resin having high molecular weight and capable of milling dispersion. The aqueous dispersed emulsion resin having a weight-average: molecular weight (Mw) of 100,000 to 300,000 may be used, and more preferably, the aqueous dispersed emulsion resin may be at least one selected from an aqueous dispersed polyurethane resin, an aqueous dispersed acrylic resin, an aqueous dispersed polyurethane acrylic resin, and an aqueous dispersed styrene acrylate resin having a weight-average molecular weight (Mw) of 100,000 to 300,000 are used.

Next, the step S50 of preparing the particulate dispersion by participating and dispersing the second pigment mixture in the step S40 with a milling machine is performed. When the second pigment mixture in the step S40 is introduced into the milling machine and particulated and dispersed, it is preferable to cool the contents by using a cooler such that the temperature of the contents does not exceed 45° C. The milling machine is preferably a basket mill, a dispersion time is preferably 3 hours to 8 hours, and an average particle size of particles obtained by particulating and dispersing the second pigment mixture is preferably 0.1 μm to 2 μm.

Next, the step S60 of filtering the particulate dispersion in the step S50 with a housing filter to prepare the aqueous dispersed color composition is performed. In the step S60, the particulate dispersion in the step S50 is filtered twice with the housing filter to remove particles having particle size distribution between D90 and D100 among the particles present in the contents to prepare; the aqueous dispersed color composition. The filtered aqueous dispersed; color composition is aged at a room temperature and at a humidity of 50% to 60% to electrostatically stabilize the aqueous dispersed pigment.

The housing filter is provided with a microfiber filter paper, in which a mesh size of the microfiber filter paper is preferably 5 μm to 10 μm for first filtration, and the mesh size of the microfiber filter paper is preferably 0.2 μm to 5μm for second filtration.

The pH regulator in the steps S10 to S30 of the present invention adjusts the pH of the alkaline resin mixture, the alkaline resin aqueous solution, and the first pigment mixture. The pH regulator may be at least one selected from a citric acid, arginine, ammonia water, triethanolamine, potassium hydroxide, sodium hydroxide tromethamine, and aminomethylpropanol.

According to the preparation method of the present invention, the particle size of the pigment is reduced, so that the aqueous dispersed color composition and the aqueous nail polish composition using the aqueous dispersed color composition have improved color expression, gloss property, and coloring strength. In addition, the pigment is mixed with the alkaline resin aqueous solution and primarily dispersed, let down with the aqueous dispersed emulsion resin, and particulated and dispersed with the milling machine, so that the pigment is electrostatically stabilized, and an attractive force between the pigment particles is rendered ineffective, thus the dispersion stability and storage stability of the pigment particles contained in the aqueous dispersed color composition are improved, and the aqueous dispersed color composition may have a high pigment content.

In addition, even when the aqueous dispersed color composition of the present invention is applied to the aqueous nail polish composition, color separation does not occur in the composition, and the dispersion stability and storage stability of the pigment may be maintained. Moreover, the pigments may be easily mixed with the aqueous nail polish composition to prepare the aqueous nail polish composition by applying the aqueous dispersed color composition of the present invention to the aqueous nail polish composition, so that the productivity can be improved. In addition, the organic solvents and dispersants conventionally used for dispersing the pigment in the nail polish are not used, so that no stimulation is applied to the skin and fingernails.

The aqueous nail polish composition may be prepared by applying the aqueous dispersed color composition prepared as described above.

The aqueous nail polish composition of the present invention includes 1 wt % to 70 wt % of an aqueous dispersed polyurethane acrylate resin, 1.0 wt % to 50 wt % of a resin aqueous solution, 0.5 wt % to 45 wt % of the aqueous dispersed color composition, 0.1 wt % to 3 wt % of a surfactant, 0.1 wt % to 2 wt % of a thickener, 0.1 wt % to 2 wt % of an adhesion promoter, and 0.1 wt % to 1 wt % of an antifoarfling agent based on a total weight of the aqueous nail polish composition.

In the aqueous nail polish composition of the present invention, the resin aqueous solution allows the aqueous nail polish composition of the present invention to form a film on surfaces of nails, and controls the gloss property, adhesion property, and water resistance of the aqueous nail polish composition. The resin aqueous solution is preferably obtained by dissolving at least one selected from a urethane resin, an acrylate resin, a vinyl acetate resin, and an alkyd resin in the water.

In the aqueous nail polish composition of the present invention, the surfactant may be at least one selected from nonionic surfactants such, as oleyl alcohol, lauryl alcohol, stearyl alcohol, sorbitol, and ethylene oxide adduct, and cationic surfactants such as fatty acid metal salt, sodium alkylsulfate, alkylether sulfates, alkyl phosphates, and alkylether phosphates. Preferably, the surfactant may be at least one selected from sodium dodecylsulfonate, sodium laurylsulfate, sodium polyoxyethylene lauryl (nonyl, octyl, oleyl, stearyl, or sorbitol) ether sodium sulfate, and sodium polyoxyethylene oleyl ether phosphate.

In the aqueous nail polish composition of the present invention, the thickener serves to control the viscosity, gloss property, and hardness of the aqueous nail polish composition, and allows the aqueous nail polish composition to maintain a predetermined viscosity and to exhibit a thixotropic property indicating flowability when the aqueous nail polish composition is applied to the fingernails. The content of the thickener is preferably 0.1 wt % to 2 wt % based on the total weight of the aqueous nail polish composition. When, the content of the thickener is less than 0.1 wt %, the viscosity of the aqueous nail polish composition is lowered, so that the thixotropic property may not be obtained when the aqueous nail polish composition is applied to the surface: of the fingernails. When the content of the thickener is greater than 2 wt %, the viscosity of the composition is high, so that workability and applicability of the composition are degraded, and the degree of gloss of the aqueous nail polish composition is degraded. The thickener, may he at least one selected from a polyurethane-based thickener, cellulose derivatives, polysaccharides, vinyl acetate polymer, organic and inorganic silica, and montmorillonite.

In addition to the above components, the aqueous nail polish composition may further include 0.1 wt % to 3 wt% of an ultraviolet absorbent, a bactericide, and a film former.

Hereinafter, the present invention will be described in more detail with reference to examples. However, these examples are for illustrative purposes only, and the scope of the present invention is not limited to these examples.

### EXAMPLES 1 TO 7

### Preparation of Aqueous Dispersed Color Composition

In Examples 1 to 7, an aqueous dispersed color composition was prepared.

### Example 1

Step S10: An alkaline resin mixture was prepared by adding and dispersing 350 g of an alkali-soluble resin for 2 hours in a dropping scheme while maintaining a temperature of a double jacket reactor containing 700 g of water and 70 g of propylene glycol at 30° C. to 40° C.

Step S20: The alkaline resin aqueous solution was prepared by heating the alkaline resin mixture in the step S10 to 75° C. and maintaining at the temperature for 3 hours, heating to 85° C. to 90° C., adding ammonia water, and maintaining for 2 hours, heating to 90° C. to 95° C. and maintaining the temperature for 2 hours, cooling to 40° C. or lower, adding the ammonia water to adjust the pH to 8.5 to 9.0, and aging at a room temperature for 1 day to 3 days.

Step S30: The first pigment mixture was: prepared by adding and dispersing 4 g of the ammonia water, 100 g of the water, and 5 g of the antifoaming agent to 400 g of the alkaline resin aqueous solution in the step S20, adding and mixing 40 wt % to 75 wt % a pigment based on 500 g of a total pigment contained in the first pigment mixture while maintaining the temperature of the double jacket reactor at 30° C. to 40° C., adding a remaining amount of the pigment two times to four times for 1 hour in a dropping scheme, and adjusting the pH to 7.0 to 8.0 by adding a citric acid. Thereafter, stirring was performed for 3 hours while maintaining the temperature at 75° C. to 85° C. by heating the inside of the double jacket reactor, 6 g of the preservative was added after cooling to 45° C. or lower, and stirring was performed for 3 minutes to 10 minutes.

Step S40: The second pigment mixture was prepared by mixing 430 g of an aqueous dispersed styrene/acrylate resin having a weight-average molecular weight (Mw) of 200,000 or more in 1000 g of the first pigment mixture in the step S30.

Step S50: A particulate dispersion was prepared by adding the second pigment mixture in the step S40 to a basket mill and particulating and dispersing at 1500 rpm for 3 hours.

Step S60: The aqueous dispersed color composition was prepared by filtering the particulate dispersion in the step S50 twice with a housing filter, and aging under a condition of a room temperature and 50% to 60% humidity.

The average particle size (D50) of the aqueous dispersed color composition prepared in Example 1 was 0.578 μm.

### Examples 2 to 4

According to the composition shown in the following Table 1, except that the first pigment mixture was prepared by mixing the alkaline resin aqueous: solution with the pigment at different amounts in the step S30, the aqueous dispersed color composition was prepared in the same manner as in Example 1.

The average particle size (D50) of the aqueous dispersed color composition prepared in Example 2 was 0.521 μm, the average particle size (D50) of the aqueous dispersed color composition prepared in Example 3 was 0.641 μm, and the average particle size (D50) of the aqueous dispersed color composition prepared in Example 4 was 0.408 μm.

TABLE 1

| First pigment | | | | |
|---|---|---|---|---|
| mixture in Step | | | | |
| S30 | Example 1 | Example 2 | Example 3 | Example 4 |
| Pigment (g) | 500 | 600 | 750 | 300 |
| Alkaline resin aqueous | 400 | 300 | 150 | 600 |
| solution (g) | | | | |
| Ammonia water (g) | 4 | 4 | 4 | 4 |
| Antifoaming agent (g) | 5 | 5 | 5 | 5 |
| Preservative (g) | 6 | 6 | 6 | 6 |
| Water (g) | 100 | 100 | 100 | 100 |

### Examples 5 to 7

According to the composition shown in the following Table 2, except that the second pigment mixture was prepared by mixing the first pigment mixture with the aqueous dispersed emersion resin at different amounts in the step S40, the aqueous dispersed color composition was prepared in the same manner as in Example 1.

The average particle size (D50) of the aqueous dispersed color composition prepared in Example 5 was 0.612 μm, the average particle size (D50) of the aqueous dispersed color composition prepared in Example 6 was 0.504 μm, and the average particle size (D50) of the aqueous dispersed color composition prepared in Example 7 was 0.426 μm.

TABLE 2

| Second pigment mixture | | | | |
|---|---|---|---|---|
| in step S40 | Example 1 | Example 5 | Example 6 | Example 7 |
| First pigment mixture (g) | 1000 | 1000 | 1000 | 1000 |
| Aqueous dispersed | 430 | 300 | 650 | 850 |
| emulsion resin (g) | | | | |

### TEST EXAMPLE 1.

### Physical Properties of Aqueous Dispersed Color Composition

The water resistance, storage stability, degree of gloss, adhesion property, and drying speed of the aqueous dispersed color compositions prepared in Examples 1 to 7 were evaluated and shown in Table 4 as follows.

The water resistance was evaluated by coating a glass plate with the aqueous dispersed color compositions of Examples 1 to 7 to form a coating film, immersing the glass plate formed with the coating film in warm water at 40° C. for 1 hour, and observing changes in the coating film (whitening, swelling, separating) with naked eyes to evaluate by a 5-point scaling scheme, in which very excellent was graded as 5 points, good was graded as 4 points, normal was graded as 3 points, defective was graded as 2 points, and very defective was graded as 1 point. The higher score represents the higher water resistance.

The storage stability was evaluated by filling ⅔ of a 40 ml sample bottle with the aqueous dispersed color compositions of Examples 1 to 7, storing the sample bottle in a thermostatic chamber at 45° C. for 4 weeks, and observing the layer division, formation of precipitates, and degree of color change with naked eyes to evaluate by the 5-point scaling scheme, in which very excellent was graded as 5 points, good was graded as 4points, normal was graded as 3 points, defective was graded as 2 points, and very defective was graded as 1 point. The higher score represents the higher storage stability.

The degree of gloss was evaluated by applying the aqueous dispersed color compositions of Examples 1 to 7 to a glass plate with a film applicator to form a coating film and measuring a reflectance three times while emitting light to the coating film at a reflection angle of 60 degrees by using a glossmeter (BYK, co., Tri-glossmeter) to obtain an average value, and the higher value represents the higher degree of gloss.

The adhesion property was evaluated using a procedure of a standard test scheme (ASTM D 3359) for measuring the adhesion property by a tape test. The adhesion property was evaluated based on Table 3 as follows by applying the aqueous dispersed color compositions of Examples 1 to 7 to the glass plate with the film applicator to form the coating film, forming 100 squares of 1 mm×1 mm on the coating film by using a cross hatch cutter, attaching a transparent cellophane tape to the cut coating film while preventing air bubbles from being formed the coating film, leaving for 1 minute to 2 minutes, and pulling out the tape to measure the number of separated squares. The higher score represents the higher adhesion property.

The drying time was evaluated by applying the aqueous dispersed color compositions of Examples 1 to 7 to the glass plate with the film applicator to form the coating film having a thickness of 90 μm and measuring a time required for drying the coating film at a room temperature until a fingerprint does not appear when pressed with a finger.

TABLE 3

| | Very | | | | |
|---|---|---|---|---|---|
| Evaluation | excellent | Excellent | Normal | Defective | Very |
| score | (5) | (4) | (3) | (2) | defective (1) |
| Number of | 0 to 5 | 6 to 10 | 11 to 20 | 21 to 30 | 31 or more |
| separated | | | | | |
| squares | | | | | |
| (number) | | | | | |

TABLE 4

| | Embodiment 1 | Embodiment 2 | Embodiment 3 | Embodiment 4 | Embodiment 5 | Embodiment 6 | Embodiment 7 |
|---|---|---|---|---|---|---|---|
| Water resistance | 5 | 4 | 1 | 2 | 1 | 5 | 1 |
| Storage stability | 5 | 5 | 1 | 2 | 1 | 5 | 2 |
| Degree of gloss | 85 | 87 | 56 | 62 | 45 | 82 | 23 |
| Adhesion property | 5 | 4 | 2 | 2 | 1 | 5 | 1 |
| Drying time | 1 min | 1 min | 1 min | 2 min | 1 min | 2 min | 3 min |
| (Finger tact) | 30 sec | 24 sec | 10 sec | 12 sec | 14 sec | 15 sec | 20 sec |

As shown in Table 4, Examples 1, 2, and 6 show excellent results in evaluation categories.

Comparing Examples 1 to 4, it was found that Example 2 has a higher degree of gloss and lower water resistance than Example 1, and Example 3 has a higher degree of gloss and lower water resistance than Example 4. In other words, it was found that the degree of gloss of the aqueous dispersed color composition is improved while the water resistance is degraded as the content of the alkaline resin aqueous solution contained in the aqueous dispersed color composition increases. In addition, it was confirmed in Example 3 that the content of the alkaline resin aqueous solution contained in the aqueous dispersed color composition was insufficient, so that the dispersion stability of the pigment was degraded, and the layer division has occurred in the aqueous dispersed color composition, thereby degrading the storage stability. Further, it was confirmed in Example 4 that the content of the alkaline resin aqueous solution contained in the aqueous dispersed color composition was excessive, so that the particulating and dispersing were difficult to be performed with the milling machine, and the layer division has occurred in the aqueous dispersed color composition, thereby degrading the storage stability.

Comparing Examples 2 and 5 to 7, it was found that Example 6 has higher water resistance and adhesion property, and a lower degree of gloss than Example 2. In other words, it was found that the water resistance and adhesion property of the aqueous dispersed color composition are improved while the degree of gloss is degraded as the content of the aqueous dispersed emulsion resin contained in the aqueous dispersed color composition increases.

In case of the drying time, it was found that the formation of the coating film was: fastest in Examples 3 and 5. In other words, it was found that the drying speed becomes faster as the content of the alkaline resin aqueous solution or the aqueous dispersed emulsion resin contained in the aqueous dispersed color composition becomes lower.

### PREPARATION EXAMPLE

### Preparation of Aqueous Nail Polish Composition

The aqueous nail polish composition was prepared by mixing 15.0 wt % of the aqueous dispersed color composition of Example 1, 60 wt % of an aqueous dispersed polyurethane acrylate resin, 20.0 wt % of the aqueous resin solution, 2.0 wt % of the surfactant, 1.0 wt % of the thickener, 1.5 wt % of the adhesion promoter, and 0.5 wt % of the antifoaming agent.

### TEST EXAMPLE 2

### Evaluation of Degree of Gloss and Viscosity of Aqueous Nail Polish Composition

In order to evaluate the viscosity and the degree of gloss of the aqueous nail polish composition prepared in Preparation Example, the viscosity was measured using a viscometer (Brookfleld DV-E viscometer), the degree of gloss was evaluated in the same manner as in Test Example 1, and results thereof are shown in Table 5 as follows.

TABLE 5

| | Aqueous nail polish composition of |
|---|---|
| | Preparation Example |
| Degree of gloss (60°) | 84 to 85 |
| Viscosity (CPS) | 1200 to 1700 |

As shown in Table 5, even when the aqueous dispersed color composition of Example 1 was applied to the aqueous nail polish composition, the degree of gloss was maintained.

### TEST EXAMPLE 3

### Evaluation of Adhesion Property of aqueous Nail Polish Composition

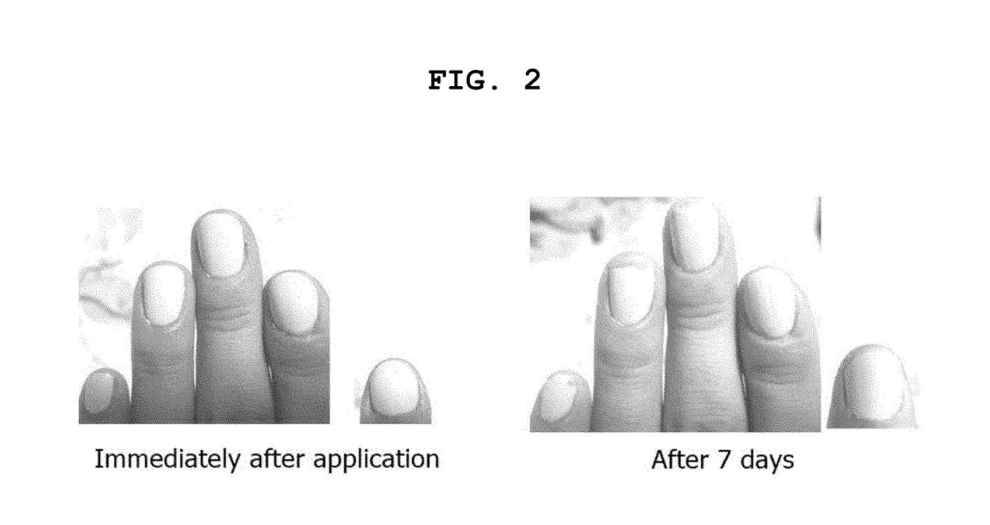

In order to confirm the adhesion property of the aqueous nail polish composition prepared in Preparation Example, the aqueous nail polish composition of Preparation Example was applied to a fingernail of a testee and the testee was left in a daily life. Then, a state of the fingernail was observed with naked eyes after one week, and results thereof are shown in FIG. 2.

As shown in FIG. 2, it was found that the color of the aqueous nail polish composition was maintained even after one week, and the coating film of the aqueous nail polish composition was maintained as a whole without breakage. In other words, it was found that the aqueous nail polish composition containing the aqueous dispersed color composition according to the present invention was excellent in the dispersibility and stability of the pigment, so that the color of the aqueous nail polish composition was hardly changed, and the aqueous nail polish composition was excellent in the adhesion property because the aqueous nail polish composition was closely adhered to the fingernail.

### TEST EXAMPLE 4

### Evaluation of Storage Stability of Aqueous Nail Polish Composition

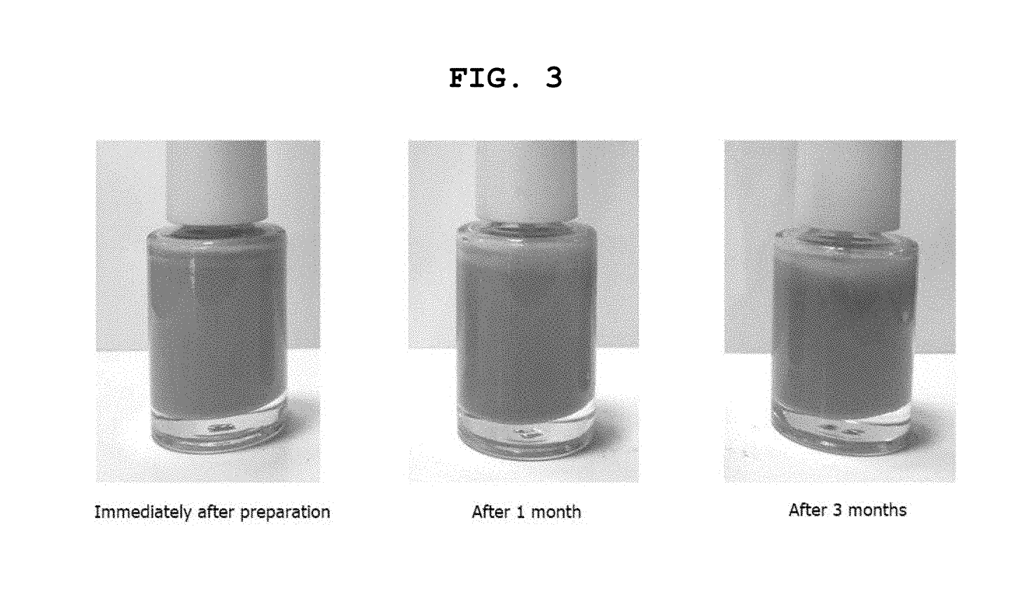

In order to confirm the storage stability of the aqueous nail polish composition prepared in Preparation Example, the color change and the layer division were observed immediately after, one month after, and three months after the preparation of the aqueous nail polish composition of Preparation Example with naked eyes, and results thereof are shown In FIG. 3.

As shown in FIG. 3, It was found that the aqueous nail polish composition containing the aqueous dispersed color composition according to the present invention is excellent in the dispersibility and stability of the pigment, so that the layer division or the color separation did not occur even when the aqueous nail polish composition was stored for a long period of time, and the storage stability was excellent.

The present invention is not limited to the above-described embodiments, and various changes and modifications can be made by those skilled in the art, which fail within the spirit and scope of the present invention as defined in the appended claims.

1. A method of preparing an aqueous dispersed color composition with a high pigment content, the method comprising:
a step (S10) of preparing an alkaline resin mixture by mixing water with an alkali-soluble resin;
a step (S20) of preparing an alkaline resin aqueous solution by aging the alkaline resin mixture in the step (S10);
a step (S30) of preparing a first pigment mixture by dispersing and emulsifying a pigment in the alkaline resin aqueous solution in the step (S20);
a step (S40) of preparing a second pigment mixture by mixing an aqueous dispersed emulsion resin with the first pigment mixture in the step (S30);
a step (S50) of preparing a particulate dispersion by particulating and dispersing the second pigment mixture in the step (S40) with a milling machine; and
a step (S60) of filtering the particulate dispersion in the step (S50) with a housing filter,
wherein the step (S30) comprises adding and dispersing a pH regulator, the water, and an antifoaming agent to the alkaline resin aqueous solution in the step (S20), adding and mixing 40 wt % to 75 wt % of the pigment contained in the first pigment mixture based on a total weight of the pigment while maintaining a temperature of a double jacket reactor at 30° C. to 40° C., adding a remaining amount of the pigment two times to four times for 1 hour in a dropping scheme, adjusting a pH to 7.0 to 8.0 by adding the pH regulator and stirring for 3 hours while maintaining the temperature at 75° C. to 85° C. by heating an inside of the double jacket reactor, adding a preservative after cooling to 45° C. or lower, and stirring for 3 minutes to 10 minutes.

2. The method of claim 1, wherein the step (S20) includes completely dissolving the alkali-soluble resin by heating the alkaline resin mixture to 60° C. to 80° C. and maintaining the temperature for 2 hours to 5 hours, heating the alkaline resin mixture to 85° C. to 90° C., adding the pH regulator, and maintaining the temperature for 1 hour to 3 hours, and heating the alkaline resin mixture to 90° C. to 95° C. and maintaining the temperature for 1 hour to 3 hours.

3. The method of claim 1, wherein, in the step (S50), an average particle size of particles obtained by particulating and dispersing the second pigment mixture in the step (S40) is 0.1 μm to 2 μm.

4. An aqueous dispersed color composition prepared by claim 1.

5. An aqueous dispersed color composition with a high pigment content, the aqueous dispersed color composition comprising:
60 wt % to 70 wt % of a first pigment mixture; and
30 wt % to 40 wt % of an aqueous dispersed emulsion resin,
wherein the first pigment mixture comprises 20 wt % to 40 wt % of an alkaline resin aqueous solution, 40 wt % to 75 wt % of a pigment, 0.1 wt % to 1.5 wt % of a pH regulator, 0.5 wt % to 2.5 wt % of a preservative, 0.2 wt % to 1.0 wt % of an antifoaming agent, and water as a remainder based on a total weight of the first pigment mixture,
the aqueous dispersed emulsion resin comprises a water-soluble resin having a weight-average molecular weight of 100000 to 300000, and
the alkaline resin aqueous solution comprises 20 wt % to 55 wt % of an alkali-soluble resin, 40 wt % to 75 wt % of the water, 0.1 wt % to 5.0 wt % of the pH regulator, and 1 wt % to 20 wt % of glycols based on a total weight of a composition of the alkaline resin aqueous solution.

6. to aqueous nail polish composition comprising the aqueous dispersed color composition prepared by claim 1.

7. An aqueous dispersed color composition prepared by one of claim 2.

8. An aqueous dispersed color composition prepared by one of claim 3.

9. An aqueous nail polish composition comprising the aqueous dispersed color composition prepared by claim 5.
